(19) United States
(12) Patent Application Publication
(10) Pub. No.: US 2013/0053639 A1
(43) Pub. Date: Feb. 28, 2013

(54) Surgical Device for Wiping Camera Lenses

(71) Applicant: Glenn M. Ihde, II, Red Oak, TX (US)

(72) Inventor: Glenn M. Ihde, II, Red Oak, TX (US)

(21) Appl. No.: 13/217,232

(22) Filed: Aug. 24, 2011

(51) Int. Cl. A61B 1/04

(52) U.S. Cl. 600/109

(57) ABSTRACT

A method of wiping a camera lens inside a human body is provided. The method comprises providing a surgical device that comprises a rod and at least one supported pad coupled to the rod and inserting the surgical device into the human body through a trocar. The method further comprises wiping the camera lens on the at least one supported pad of the surgical device while the surgical device is inside the human body and removing the surgical device from the human body.

### CROSS-REFERENCE TO RELATED APPLICATIONS

Not applicable.

### STATEMENT REGARDING FEDERALLY SPONSORED RESEARCH OR DEVELOPMENT

Not applicable.

### REFERENCE TO A MICROFICHE APPENDIX

Not applicable.

### BACKGROUND

Laparoscopic surgery is a form of minimally invasive, endoscopic surgery performed in the abdomen. With laparoscopic surgery, general anesthesia is given. A small incision is usually made below or inside the umbilicus. The abdomen is then insufflated with an inert gas, such as carbon dioxide, by inserting a special needle or a trocar through the umbilicus. A trocar, also called a trocar sleeve or cannula, is a specially designed tube, usually 5 or 10 mm in diameter, with a valve through which a surgeon can insert special instruments. Insufflation induces a state of pneumoperitoneum, which enhances the surgeon's view and ability to make manipulations. If a special needle is used, the surgeon removes the needle after an adequate volume of gas is insufflated into the abdominal cavity and inserts a trocar. The valve in the trocar prevents the inert gas from escaping through the trocar. This helps to maintain the state of pneumoperitoneum. An endoscope, also called a laparoscope, is then placed through the trocar. The laparoscope allows the surgeon to visualize the pelvic and abdominal organs on a video monitor. Additional smaller incisions are made in the abdomen to allow placement of additional trocars as needed. The additional trocars are for the surgeon to place specially designed surgical instruments into the abdominal cavity, allowing the surgeon to carry out the same procedure as in open surgery.

For patients, the advantages of laparoscopic surgery over open surgery include reduced trauma to the body, reduced blood loss, and smaller surgical scars. Patients also leave the hospital sooner after laparoscopic and return to normal activities sooner than with conventional open surgery. Similar minimally invasive approaches, such as thoracoscopic surgery, may be performed on other areas of the body. These approaches share some of the same advantages and challenges as laparoscopic surgery.

### SUMMARY

In one embodiment of the disclosure, a method of wiping a camera lens inside a human body is provided. The method comprises providing a surgical device that comprises a rod and at least one supported pad coupled to the rod and inserting the surgical device into the human body through a trocar. The method further comprises wiping the camera lens on the at least one supported pad of the surgical device while the surgical device is inside the human body and removing the surgical device from the human body.

In another embodiment of the disclosure, a system for wiping a camera lens inside a human body is provided. The system comprises at least one trocar, a camera comprising a lens, and a surgical device comprising a rod with at least two ends and a supported pad at a first end of the rod. After the surgical device is inserted into the at least one trocar and the supported pad of the surgical device is passed through the distal end of the at least one trocar into the human body, the lens of the camera is wiped on the supported pad of the surgical device.

In yet another embodiment of the disclosure, a surgical device that wipes a camera lens inside a human body is provided. The surgical device comprises a rod having at least two ends and a pad coupled to a first end of the rod. The pad is supported by the rod. The surgical device also comprises a movable cover coupled to the first end of the rod. The movable cover protects the pad.

These and other features will be more clearly understood from the following detailed description taken in conjunction with the accompanying drawings and claims.

### BRIEF DESCRIPTION OF THE DRAWINGS

For a more complete understanding of this disclosure, reference is now made to the following brief description, taken in connection with the accompanying drawings and detailed description, wherein like reference numerals represent like parts.

FIGS. 1A and 1B illustrate a surgical device according to an embodiment of the disclosure.

FIGS. 2A and 2B illustrate a surgical device according to another embodiment of the disclosure.

FIGS. 3A to 3E illustrate a surgical device according to an additional embodiment of the disclosure.

FIG. 4 illustrates a surgical device according to a further embodiment of the disclosure.

FIG. 5 illustrates a method of wiping a camera lens inside a human body.

### DETAILED DESCRIPTION

It should be understood at the outset that although illustrative implementations of one or more embodiments are illustrated below, the disclosed systems and methods may be implemented using any number of techniques, whether currently known or not yet in existence. The disclosure should in no way be limited to the illustrative implementations, drawings, and techniques illustrated below, but may be modified within the scope of the appended claims along with their full scope of equivalents.

The key element in laparoscopic surgery is the use of a laparoscope to view and illuminate the operative field. The laparoscope comprises a telescopic rod lens system that is connected to a video camera (single chip or three chip). The rod is made of a light conducting material that is used to pass the image from the tip of the rod to the camera head. The rod is passed through the trocar and into the abdominal cavity where it transmits the light and images from inside the abdomen to the camera. The camera then transmits the light and images to a video monitor for viewing.

When the laparoscope passes through the trocar, fluid materials deposited on the trocar during insertion have a tendency to pass onto the lens of the laparoscope and obscure the view of the camera. Fluid materials can pass onto the lens as it passes through a valve. Fluid materials can also pass onto the lens, for example, as it passes through the inner walls of the trocar. Fluid materials along the inner walls and the tip of the trocar can transfer to the lens by way of capillary action due to the fluid material's surface tension. Also, fluid materials can pass onto the lens while the laparoscope is moved around inside the body.

Previously, when fluid materials obscured the lens, the laparoscope would have to be removed from inside the trocar, and the lens would have to be cleaned outside of the abdominal cavity, which increases the total time that it takes to perform the laparoscopy. This in turn increases the amount of time that the patient is under anesthesia as well as the amount of time the instruments are inside the abdominal cavity without being directly visualized.

Accordingly, the pending disclosure teaches a surgical device that enables the laparoscopic lens to be wiped while inside the human body. In an embodiment, the surgical device comprises a rod having at least two ends and a pad coupled to a first end of the rod such that the pad is supported by the rod. The surgical device may also comprise a movable cover that protects the pad as the surgical device is inserted into a trocar. After the surgical device is inserted through the trocar into the human body and before a camera lens is brought into contact with the pad, the movable cover may be moved. Having a movable cover as a part of the surgical device may protect the pad and keep the pad clean and dry as the pad is inserted through the trocar into a human body.

Furthermore, in an embodiment, the end of the rod of the surgical device that comprises the pad is adjustable to a plurality of angles. The ability to adjust the end of the rod of the surgical device that comprises the pad may enable the camera lens to make more effective contact with the pad at an angle more conducive for wiping the camera lens.

If a camera lens is cleaned outside the human body as was previously done, there is a risk that as the camera lens is reinserted through the trocar, the lens may become dirty again. Such a risk is eliminated with the disclosed surgical device because the surgical device enables the cleaning of the camera lens inside the human body. Additionally, since the surgical device of the disclosure enables the laparoscopic lens to be wiped while inside the human body instead of having to remove the laparoscopic lens from inside the trocar and wipe the lens outside of the abdominal cavity, the total time it takes to perform the laparoscopy is reduced, which reduces the amount of time that the patient is under anesthesia. Further, since the surgical device of the disclosure enables wiping of the laparoscopic lens while inside the human body, the surgeon is able to receive instantaneous feedback regarding whether or not the lens is free from obscurities. Moreover, the wiping of the laparoscopic lens on the surgical device disclosed herein while inside the human body may result is a cleaner lens, thereby enabling greater precision in the performance of the surgery at hand.

Although the use of the surgical device of the disclosure is described in the context of laparoscopic surgery, one of ordinary skill in the art would recognize that it could also be used in other endoscopic or minimally invasive procedures, such as thoracoscopic surgery.

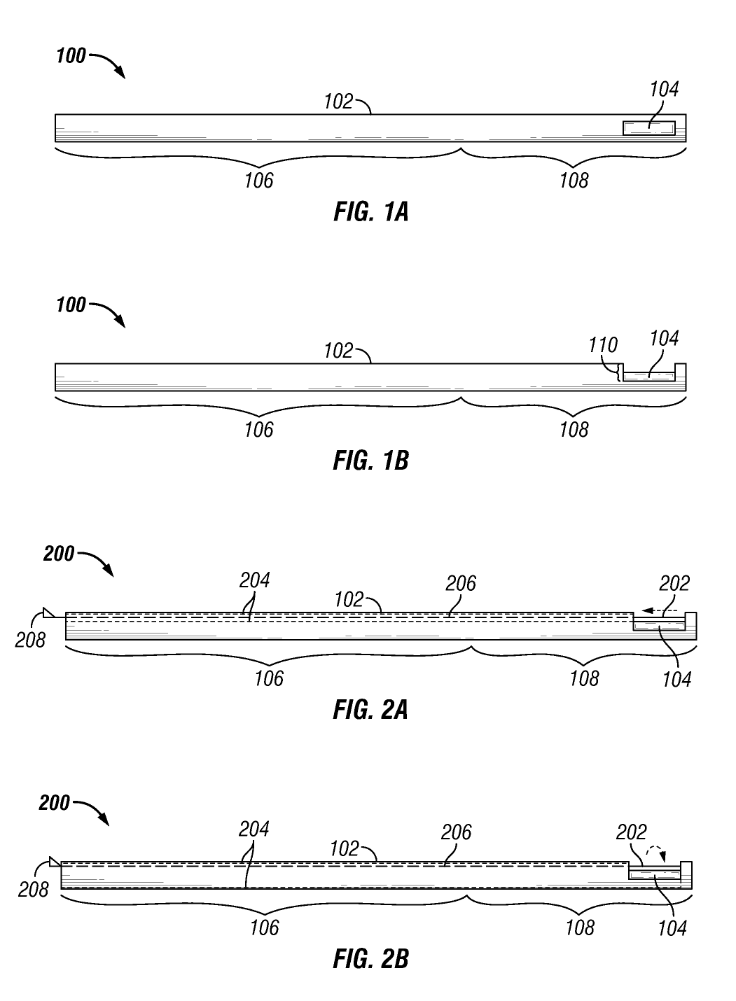

FIGS. 1A and 1B illustrate an embodiment of a lens wiping surgical device 100. The lens wiping surgical device 100 may comprise a rod 102 and a pad 104. The rod 102 may be of a shape and size such that the rod 102 can be inserted into a human body through a trocar. In an embodiment, the rod 102 is made of a type of plastic that is commonly used in many surgical instruments. The rod 102 is shown as having a first part 106 and a second part 108.

In an embodiment, the pad 104 is coupled to the second part 108 of the rod 102. Coupled as used herein means directly or indirectly connected. In such an embodiment, the pad 104 is supported by the rod 102. Supporting the pad 104 with the rod 102 provides a backstop that reinforces the pad 104 and helps lessen or prevent the backward movement of the pad 104 as the camera lens is wiped thereon.

The pad 104 may be made of sterile cotton, foam, or another type of absorbent sterile material. For example, the pad 104 may be made of polyvinyl acetyl (PVA) foam. In an embodiment, the pad 104 is of a shape and size such that at least a portion of a camera lens, such as a camera lens from a laparoscope or an endoscope, can be wiped on the pad 104. While the pad 104 is illustrated in FIGS. 1A and 1B as being rectangular in shape, one of ordinary skill in the art will appreciate that the pad 104 could take a number of different shapes without departing from the scope of the disclosure. For example, the pads 104 may be circular, parabolic, square, or another shape.

In an embodiment, the pad 104 may be inset in a cavity 110. As discussed above, fluid materials often become deposited on the inside surfaces of a trocar. Insetting the pad 104 in the cavity 110 may protect the pad 104 and keep the pad 104 clean and dry as the pad 104 is inserted through the trocar into a human body. In embodiments where the pad 104 is inset in a cavity, the pad 104 may be larger than the camera lens itself.

In alternative embodiments, the pad 104 may not be inset within a cavity, but rather may be flush with or protrude beyond the surface of the rod 102. Making the pad 104 flush with or protrude beyond the surface of the rod 102 may enable easier wiping of the camera lens. In such alternative embodiments, the pad 104 may be smaller than the camera lens itself. While shown as flat, in other embodiments, the pad 104 could be convex or another shape.

In an embodiment, the lens wiping surgical device 100 is a one time use product. For instance, after the lens wiping surgical device 100 is inserted through a trocar into a human body, a camera lens is wiped against the pad 104 of the lens wiping surgical device 100, and the lens wiping surgical device 100 is removed from the human body. If the camera lens requires wiping later on during the surgical procedure, a new lens wiping surgical device 100 may be used. Such an embodiment would eliminate the risks associated with reinserting a device into the human body once it has been removed.

Alternatively, the lens wiping surgical device 100 may be a multi-use product. For example, the second part 108 of the rod 102 may be detachable from the first part 106 of the rod 102, thereby enabling replacement of the second part 108 of the rod 102 with a new second part after use. In an embodiment, the first part 106 and the second part 108 of the rod 102 may comprise threads to enable detachment of the second part 108 of the rod 102 from the first part 106 of the rod 102. For instance, the second part 108 of the rod 102 may screw on and off or into and out of the first part 106 of the rod 102. One of ordinary skill in the art will appreciate that detachment of the second part 108 of the rod 102 from the first part 106 of the rod 102 may be enabled in a number of other ways besides the use of threads without departing from the scope of the disclosure.

Turning now to FIGS. 2A and 2B, an embodiment of a lens wiping surgical device 200 is illustrated. In an embodiment, the lens wiping surgical device 200 is similar to the lens wiping surgical device 100 illustrated in FIGS. 1A and 1B except that the lens wiping surgical device 200 also comprises a movable cover 202. As stated above, fluid materials often become deposited on the inside surfaces of a trocar. Having a movable cover 202 as a part of the lens wiping surgical device 200 may protect the pad 104 and keep the pad 104 clean and dry as the pad 140 is inserted through the trocar into a human body. The movable cover 202 may be more beneficial when the pad 104 is flush with or protrudes beyond the surface of the rod 102. However, as illustrated in FIGS. 2A and 2B, the movable cover 202 may still be present even when the pad 104 is inset in a cavity.

The movable cover 202 may be made of plastic, such as a type of plastic that is commonly used in many surgical instruments, or another type of material. In an embodiment, the movable cover 202 covers the pad 104 as the lens wiping surgical device 200 is inserted through a trocar into a human body. Once inside the human body, the movable cover 202 may be moved, thereby exposing the pad 104 and enabling a camera lens to be wiped on the pad 104. In an embodiment, the movable cover 202 is retractable as illustrated in FIG. 2A. In another embodiment, the movable cover 202 is rotatable as illustrated in FIG. 2B.

In order to enable the movement of the movable cover 202, the lens wiping surgical device 200 may comprise a channel 204, a cable 206, and an actuator 208. In an embodiment, the channel 204 is of a size and shape such that the cable 206 can fit within the channel 204. Also, the channel 204 may be of a size and shape such that the movable cover 202 can retract into the channel 204. For instance, in the embodiment of FIG. 2A, the channel 204 runs along the length of the rod 102. In the embodiment of FIG. 2B, the channel 204 may run along the perimeter or a portion of the perimeter of the rod 102.

The cable 206 may be coupled to the movable cover 202. In an embodiment, the cable 206 is made of plastic, metal, nylon, or another material. The cable 206 may be housed in the channel 204. In an embodiment, such as in the embodiment of FIG. 2A, a portion of the cable 206 may be outside the lens wiping surgical device 200 when the cable 206 is actuated, such as by use of the actuator 208. In other embodiments, such as in the embodiment of FIG. 2B, the cable 206 remains within the channel 204 regardless of whether or not the cable 206 is actuated.

The actuator 208 may be located at or near the end of the rod 102. In an embodiment, the actuator 208 may be coupled to the cable 206. The actuator 208 may be movable and/or rotatable. For example, in the embodiment of FIG. 2A, the actuator 208 may be movable forward and backward by a user's thumb and/or other finger(s). In another example, such as in the embodiment of FIG. 2B, the actuator 208 may be rotatable left and right or forward and backward by a user's thumb and/or other finger(s).

After the lens wiping surgical device 200 is inserted through a trocar into the human body and before a camera lens is brought into contact with the pad 104, the movable cover 202 may be moved. Referring to FIG. 2A, the movable cover 200 may retract into the lens wiping surgical device 200 by moving the actuator 208, such as by pulling back on the actuator 208. In an embodiment, such an action causes the cable 206 to pull back the movable cover 202 into the channel 204, thereby exposing a clean and dry pad 104. Referring to FIG. 2B, the movable cover 202 may be rotated into the lens wiping surgical device 200 by moving the actuator 208, such as by rotating the actuator 208. In an embodiment, such an action causes the cable 206 to rotate the movable cover 202 into the channel 204, thereby exposing a clean and dry pad 104.

In alternative embodiments, rather than the movable cover 202 retracting or rotating into the lens wiping surgical device 200, the movable cover 202 may retract or rotate onto the surface of the rod 102. While the movable cover 202 is illustrated in FIG. 2A as being retractable and in FIG. 2B as being rotatable, one of ordinary skill in the art will appreciate that movement of the movable cover 202 may be enabled in a number of other ways without departing from the scope of the disclosure.

In an embodiment, the movable cover 202 may move or slide in multiple directions so as to act as more than just a pad covering component. For example, the movable cover 202 may also act as a squeegee component while the lens wiping surgical device 200 is inside the human body. In an embodiment, in addition to covering the pad 104 as the lens wiping surgical device 200 is inserted into the trocar, the movable cover 202 may also squeeze or apply pressure to the pad 104, thereby eliminating moisture in the pad 104.

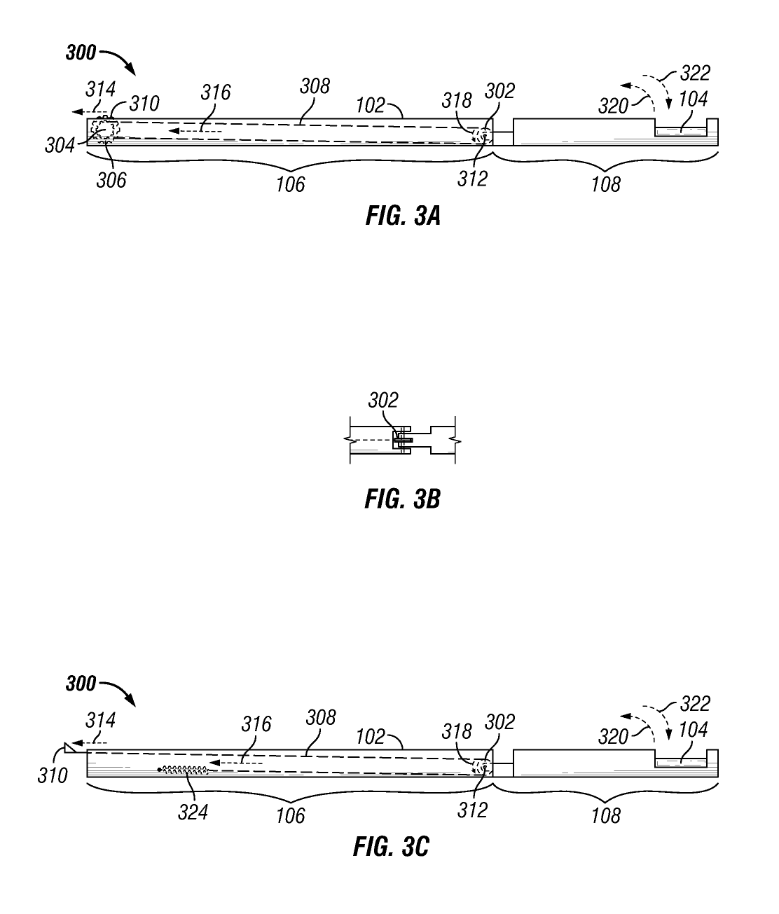

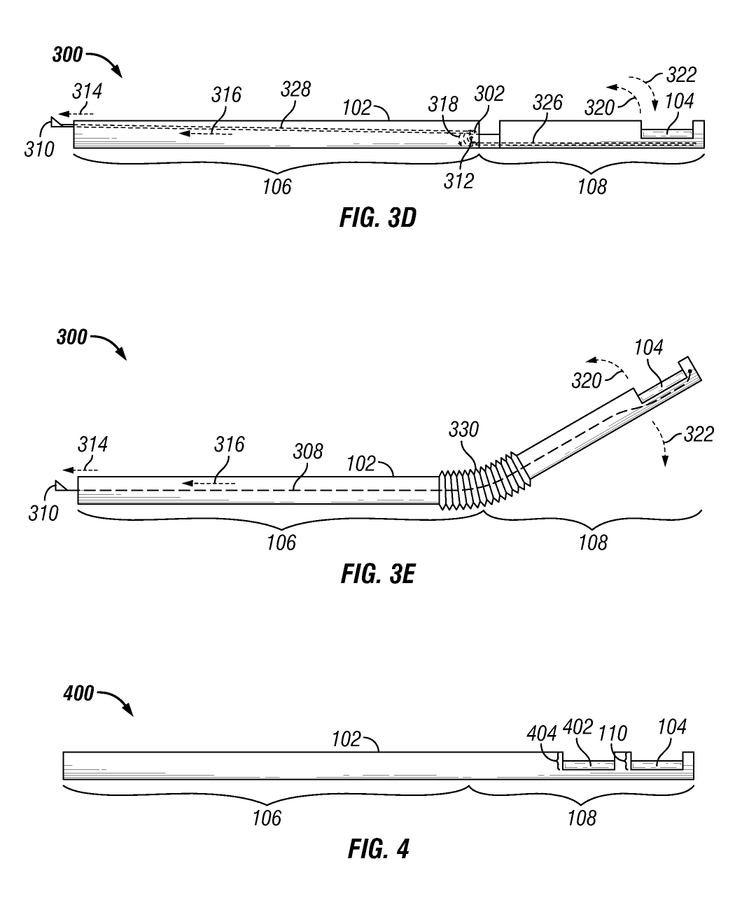

Turning now to FIGS. 3A to 3E, an embodiment of a lens wiping surgical device 300 is illustrated. In an embodiment, the lens wiping surgical device 300 is similar to the lens wiping surgical device 100 illustrated in FIGS. 1A and 1B except that the second part 108 of the rod 102 of the lens wiping surgical device 300 is adjustable. For example, the second part 108 of the rod 102 may be adjustable to a plurality of angles. Depending on the placement of the trocar through which the laparoscope or endoscope is inserted, the position and/or angle of the laparoscope or endoscope, and other factors, the camera lens on the laparoscope or endoscope may not be able to make the most effective contact with the pad 104 of the rigid lens wiping surgical device 100 illustrated in FIGS. 1A and 1B. Under such circumstances, adjusting the second portion 108 of the rod 102 while inside the human body may enable the camera lens to be brought into improved contact with the pad 104 of the adjustable lens wiping surgical device 300 illustrated in FIGS. 3A to 3E. Furthermore, even if the camera lens on the laparoscope or endoscope were able to make contact with the pad 104 of the rigid lens wiping surgical device 100 illustrated in FIGS. 1A and 1B, adjusting the second portion 108 of the rod 102 while inside the human body may enable the camera lens to make better contact with pad 104 at an angle more conducive for wiping the camera lens.

As is illustrated in FIGS. 3A-3E, the adjustment of the second part 108 of the rod 102 may be enabled in a number of different ways. For example, as illustrated in FIGS. 3A and 3B, the lens wiping surgical device 300 may comprise a pulley system to enable adjustment of the second part 108 of the rod 102. FIG. 3A is a side view and FIG. 3B is a top view of the lens wiping surgical device 300 comprising a pulley system. The pulley system may comprise a first wheel 302, a second wheel 304, a third wheel 306, a cable 308, an actuator 310, and a pivot point 312.

The second part 108 of the rod 102 may be coupled to the first wheel 302. In an embodiment, the first part 106 of the rod 102 comprises a second wheel 304 and third wheel 306. The first wheel 302, the second wheel 304, and/or the third wheel 306 may comprise teeth. In an embodiment, a portion of the second wheel 304 may be inset in the third wheel 306 such that movement of the third wheel 306 causes the second wheel 304 to move as well. A cable 308 may be coupled to the first wheel 302 and the second wheel 304. In an embodiment, the cable 308 may be made of plastic, metal, nylon, or another material. At least a portion of the cable 308 may comprise a chain.

An actuator 310 may be located on one of the sides of the rod 102 toward the end of the rod 102. The actuator 310 may be a thumb screw, a lever, a handle, or another component capable of activating the pulley system. The actuator 310 may be movable and/or rotatable by a user's thumb and/or other finger(s). In the embodiment illustrated in FIG. 3A, a portion of the third wheel 306 is exposed such that the actuator 310 is a thumb screw that is rotatable forward and backward.

In an embodiment, movement of the actuator 310 actuates the pulley system, thereby causing the second part 108 of the rod 102 to adjust to a plurality of angles about the pivot point 312. For example, referring to FIG. 3A, if a user rotates the actuator 310 backward (such as in the direction of arrow 314), the third wheel 308 will rotate backward, thereby causing the second wheel 304 to rotate backward, which in turn causes the cable 308 to move backward (such as in the direction of arrow 316), the first wheel 302 to rotate backward (such as in the direction of arrow 318), and the second part 108 of the rod 102 to rotate in the direction of arrow 320 about the pivot point 312. If the user wants the second part 108 of the rod 102 to move in the opposite direction of arrow 320 about the pivot point 312 (such as in the direction of arrow 322), the user may rotate the actuator 310 forward.

In an embodiment, as illustrated in FIG. 3C, the pulley system may be implemented using a spring 324 instead of the second and third wheels 304 and 306. The spring 324 may be coupled to the first part 106 of the rod 102 and the cable 308. The actuator 310 may also be coupled to the cable 308 and may be capable of activating the lever system. In an embodiment, as a user pulls back on the actuator 310 (such as in the direction of arrow 314), the cable 308 rotates backward (such as in the direction of arrow 316), thereby causing the first wheel 302 to rotate backward (such as in the direction of arrow 318), the spring 324 to expand, and the second part 108 of the rod 102 to rotate in the direction of arrow 320 about the pivot point 312. If the user wants the second part 108 of the rod 102 to move in the opposite direction of arrow 320 about the pivot point 312 (such as in the direction of arrow 322), the user may release the actuator 310.

In another example, as illustrated in FIG. 3D, the lens wiping surgical device 300 may comprise a lever system to enable adjustment of the second part 108 of the rod 102. The lever system may comprise the first wheel 302, a first rod 326, a second rod 328, the actuator 310, and the pivot point 312. In an embodiment, the first rod 326 and/or the second rod 328 are made of made of plastic, metal, or another material. The first rod 326 may be coupled to the first wheel 302 and the second part 108 of the rod 102 and the second rod 328 may be coupled to the first wheel 302 and the first part 106 of the rod 102. While described as being coupled to the first wheel 302, the first rod 326 and the second rod 328 may instead be coupled to rectangular piece or a component of a different shape. In an embodiment, the actuator 310 is coupled to the second rod 328.

Movement of the actuator 310 may actuate the lever system, thereby causing the second part 108 of the rod 102 to adjust to a plurality of angles about the pivot point 312. For example, referring to FIG. 3D, if a user pulls the actuator 310 backward (such as in the direction of arrow 314), the second rod 328 will rotate backward (such as in the direction of arrow 316), thereby causing the first wheel 302 to rotate backward (such as in the direction of arrow 318) and the first rod 326 to rotate backward (such as in the direction of arrow 320), which in turn causes the second part 108 of the rod 102 to rotate in the direction of arrow 320 about the pivot point 312. If the user wants the second part 108 of the rod 102 to move in the opposite direction of arrow 320 about the pivot point 312 (such as in the direction of arrow 322), the user may move the actuator 310 in the opposite direction of arrow 314.

In yet another example, as illustrated in FIG. 3E, the lens wiping surgical device 300 may comprise a premolded joint system to enable adjustment of the second part 108 of the rod 102. The premolded joint system may comprise a flexible joint 330, the cable 308, and the actuator 310. In an embodiment, the flexible joint 330 is located between the first part 106 of the rod 102 and the second part 108 of the rod 102. The flexible joint 330 may be made of a flexible material to enable the second part 108 of the rod 102 to adjust to a plurality of angles. For example, the flexible joint 330 may be made of a flexible plastic, rubber, or another flexible material.

The cable 308 may be coupled to the second part 108 of the rod 102 and the actuator 310. In an embodiment, movement of the actuator 310 actuates the premolded joint system thereby causing the second part 108 of the rod 102 to adjust to a plurality of angles. For example, referring to FIG. 3E, if a user moves the actuator 310 backward (such as in the direction of arrow 314), the cable 308 will move backward (such as in the direction of arrow 316), thereby causing the flexible joint 330 to straighten and the second part 108 of the rod 102 to rotate in the direction of arrow 322. As the user releases the actuator 310, the flexible joint 330 may curve and the second part 108 of the rod 102 may rotate in the direction of arrow 320.

While the adjustment of the second part 108 of the rod 102 is described above as being enabled by a pulley system, a lever system, or premolded joint system, one of ordinary skill in the art will appreciate that the adjustment of the second part 108 of the rod 102 may be enabled in a number of other ways without departing from the scope of the disclosure. For example, the adjustment of the second part 108 of the rod 102 may be enabled by other mechanical components, electronic components, and/or hydraulic components. Furthermore, while the adjustment of the second part 108 of the rod 102 was described above in FIGS. 3A to 3E in terms of the rotation of the second part 108 of the rod 102 up and down (such as in the direction of arrows 320 and 322), the second part 108 of the rod 102 may also be rotatable from side to side (i.e., left and right).

Turning now to FIG. 4, an embodiment of a lens wiping surgical device 400 is illustrated. In an embodiment, the lens wiping surgical device 400 is similar to the lens wiping surgical device 100 illustrated in FIGS. 1A and 1B except that the lens wiping surgical device 400 comprises a second pad 402 instead of just a single pad 104. As may be the case with pad 104, the second pad 402 may be coupled to the second part 108 of the rod 102 such that the second pad 402 is also supported by the rod 102. Supporting both of the pads 104 and 402 with the rod 102 provides a backstop that reinforces the pads 104 and 402 and helps lessen or prevent the backward movement of the pads 104 and 402 as the camera lens is wiped thereon.

The pads 104 and 402 may be made of sterile cotton, foam, or another type of absorbent sterile material. For example, the pads 104 and 402 may be made of polyvinyl acetyl (PVA) foam. In an embodiment, the pads 104 and 402 are made of the same type of material. In alternative embodiments, the pads 104 and 402 are made of different types of material.

The pad 104 and 402 may be of a shape and size such that at least a portion of a camera lens, such as a camera lens from a laparoscope or an endoscope, can be wiped on the pads 104 and 402. While both of the pads 104 and 402 are illustrated in FIG. 4 as being rectangular in shape, one of ordinary skill in the art will appreciate that the pad 104 and/or the pad 402 could take a number of different shapes without departing from the scope of the disclosure. For example, the pads 104 and/or 402 may be circular, parabolic, square, or another shape.

As described above, the pad 104 may be inset in a cavity 110. Similarly, the second pad 402 may be inset in a second cavity 404. Insetting the pad 104 and/or the second pad 402 in cavities may protect the pads 104 and 402 and keep the pads 104 and 402 clean and dry as the pads 104 and 402 are inserted through the trocar into a human body.

In alternative embodiments, as described above, the pad 104 may not be inset within a cavity, but rather may be flush with or protrude beyond the surface of the rod 102. Similarly, the second pad 402 may be flush with or protrude beyond the surface of the rod 102 rather than being inset within a cavity. Making the pad 104 and/or the second pad 402 flush with or protrude beyond the surface of the rod 102 may enable easier wiping of the camera lens. While shown as flat, in other embodiments, the pads 104 and 402 could be convex or another shape. For instance, in some embodiments, the pads 104 and/or 402 may have a variable wave shape to enhance contact with the camera lens.

As described above with respect to FIGS. 1A and 1B, the lens wiping surgical device 400 may also be a one time use product. Alternatively, the lens wiping surgical device 400 may be a multi-use product. Additionally, while not illustrated in FIG. 4, the lens wiping surgical device 400 may comprise a movable cover as discussed above with respect to FIGS. 2A and 2B. In an embodiment, the movable cover is of a size and shape such that it covers both the pad 104 and the second pad 402. Alternatively, there may be multiple movable covers, one movable cover that protects the pad 104 and one movable cover that protects the second pad 402, or there may be a single movable cover that protects just one of the pads 104 and 402. Furthermore, while not illustrated in FIG. 4, the second part 108 of the rod 102 of the lens wiping surgical device 400 may be adjustable as discussed above with respect to FIGS. 3A to 3E.

In an embodiment, one of the pads 104 and 402 is considered the cleaning pad and the other of the pads 104 and 402 is considered the wiping pad. For example, pad 104 may be the cleaning pad and the second pad 402 may be the wiping pad. Alternatively, the second pad 402 may be the cleaning pad and the pad 104 may be the wiping pad.

The cleaning pad, which as described above may be either the pad 104 or the second pad 402, may comprise a cleaning substance. The cleaning substance may be in a liquid form, a powder form, a gel form, or another form. In an embodiment, the cleaning substance is a mixture of alcohol and water. Alternatively, the cleaning substance may be another substance that is safe to insert into the human body and is capable of cleaning a camera lens.

The cleaning substance may be applied to the cleaning pad before or after delivery of the lens wiping surgical device 400 to the surgical site. For example, in an embodiment, the cleaning substance is applied to the cleaning pad during manufacture. In such an embodiment, the lens wiping surgical device 400 may be sealed and then delivered to the surgical site. Alternatively, the cleaning substance may be applied prior to surgery at the surgical site. In such an embodiment, the lens wiping surgical device 400 may be manufactured with two dry pads and the cleaning substance may be applied to one of the two dry pads just prior to surgery by healthcare personnel.

As described above, the wiping pad may be either the pad 104 or the second pad 402. However, unlike the cleaning pad, the wiping pad may comprise no cleaning substance or additional substances. Instead, the wiping pad may be a dry pad.

For illustrative purposes only, assume that the second pad 402 is the cleaning pad and the pad 104 is wiping pad. In an embodiment, after the lens wiping surgical device 400 is inserted through a trocar into a human body, a camera lens is wiped against the second pad 402, which is the cleaning pad in the above example. Next, the camera lens may be wiped against the pad 104, which is the wiping pad in the above example, and then the lens wiping surgical device 400 may be removed from the human body. Having a cleaning pad and a wiping pad (e.g., pads 104 and 402) instead of just a single pad (e.g., pad 104) may enable the camera lens to be freed from even more obscurities, thereby resulting in an overall cleaner camera lens.

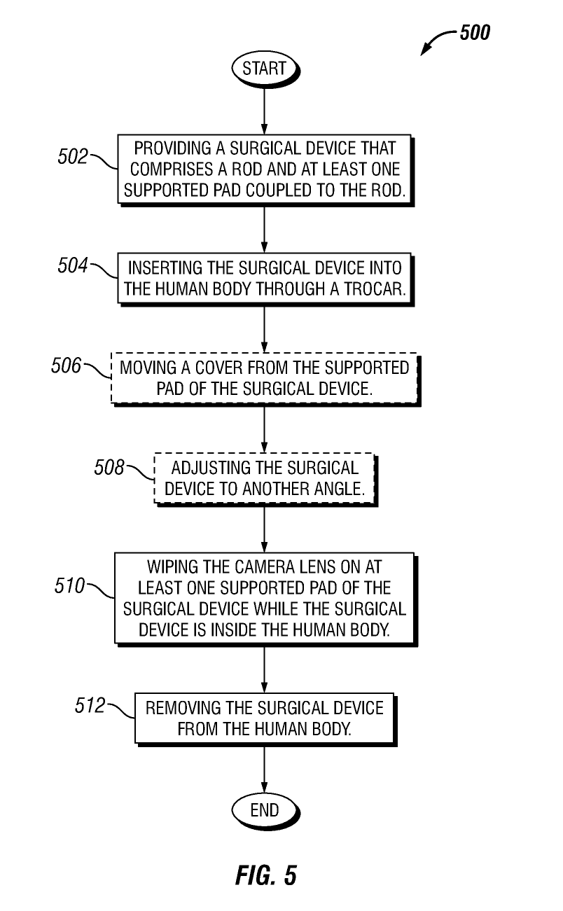

Turning now to FIG. 5, a method 500 of wiping a camera lens inside a human body is illustrated. At block 502, a surgical device that comprises a rod and at least one supported pad coupled to the rod is provided. The surgical device is then inserted into the human body through a trocar at block 504. At block 506, a cover may be moved from the supported pad of the surgical instrument. The surgical device may be adjusted to another angle at block 508. At block 510, the camera lens is wiped on the at least one supported pad of the surgical instrument while the surgical device is inside the body. Next, the surgical device is removed from the human body at block 512.

While several embodiments have been provided in the present disclosure, it should be understood that the disclosed systems and methods may be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted or not implemented.

Also, techniques, systems, subsystems, and methods described and illustrated in the various embodiments as discrete or separate may be combined or integrated with other systems, modules, techniques, or methods without departing from the scope of the present disclosure. Other items shown or discussed as directly coupled or communicating with each other may be indirectly coupled or communicating through some interface, device, or intermediate component, whether electrically, mechanically, or otherwise. Other examples of changes, substitutions, and alterations are ascertainable by one skilled in the art and could be made without departing from the spirit and scope disclosed herein.

What is claimed is:

1. A method of wiping a camera lens inside a human body, comprising:
providing a surgical device that comprises a rod and at least one supported pad coupled to the rod;
inserting the surgical device into the human body through a trocar;
wiping the camera lens on the at least one supported pad of the surgical device while the surgical device is inside the human body; and
removing the surgical device from the human body.

2. The method of claim 1, further comprising adjusting the surgical device to another angle.

3. The method of claim 2, wherein the adjusting the surgical device to another angle occurs after the inserting the surgical device into the human body through the trocar and before the wiping the camera lens on the at least one supported pad of the surgical device.

4. The method of claim 1, further comprising moving a cover from the supported pad of the surgical device.

5. The method of claim 4, wherein the moving the cover from the supported pad of the surgical device occurs after the inserting the surgical device into the human body through the trocar and before the wiping the camera lens on the at least one supported pad of the surgical device.

6. The method of claim 1, wherein the surgical device comprises two supported pads, a first of the two supported pads for cleaning the camera lens and a second of the two supported pads for wiping the camera lens.

7. The method of claim 6, wherein the wiping the camera lens on the at least one supported pad of the surgical device comprises cleaning the camera lens on the first of the two supported pads and wiping the camera lens on the second of the two supported pads.

8. The method of claim 7, wherein the cleaning the camera lens on the first of the two supported pads occurs before the wiping the camera lens on the second of the two supported pads.

9. A system for wiping a camera lens inside a human body, comprising:
at least one trocar;
a camera comprising a lens; and
a surgical device comprising a rod with at least two ends and a supported pad at a first end of the rod, wherein after the surgical device is inserted into the at least one trocar and the supported pad of the surgical device is passed through the distal end of the at least one trocar into the human body, the lens of the camera is wiped on the supported pad of the surgical device.

10. The system of claim 9, wherein the camera is part of an endoscope.

11. The system of claim 9, wherein the surgical device further comprises a rotation coupling, and wherein the rotation coupling enables the first end of the rod to adjust to a plurality of angles.

12. The system of claim 9, wherein the surgical device further comprises a movable cover, and wherein the movable cover protects the supported pad.

13. The system of claim 9, wherein the surgical device further comprises a second supported pad at the first end of the rod, and wherein after the surgical device is inserted into the at least one trocar and the second supported pad of the surgical device is passed through the distal end of the at least one trocar into the human body, the second supported pad of the surgical device comes into contact with and cleans the lens of the camera.

14. A surgical device that wipes a camera lens inside a human body, comprising:
a rod having at least two ends;
a pad coupled to a first end of the rod, wherein the pad is supported by the rod; and
a movable cover coupled to the first end of the rod, wherein the movable cover protects the pad.

15. The surgical device of claim 14 further comprising a rotation coupling located on the rod between the first end of the rod and a second end of the rod, wherein the rotation coupling enables the first end of the rod to adjust to a plurality of angles.

16. The surgical device of claim 14, wherein the movable cover is retractable or rotatable.

17. The surgical device of claim 14, wherein the first end of the rod comprises a cavity, and wherein the cavity contains the pad.

18. The surgical device of claim 17 further comprising a second pad coupled to the first end of the rod, wherein the second pad is supported by the rod, wherein the first end of the rod further comprises a second cavity, and wherein the second cavity contains the second pad.

19. The surgical device of claim 18, wherein the second pad comprises a cleaning substance.

20. The surgical device of claim 14, wherein the pad is made of sterile cotton or foam.
